(19) United States
(12) Patent Application Publication
(10) Pub. No.: US 2007/0135816 A1
(43) Pub. Date: Jun. 14, 2007

(54) Bone anchor

(75) Inventors: Philip Kropf, Pfaffikon (CH); Jan Leuzinger, Pfaffikon (CH)

Correspondence Address: MARSHALL, GERSTEIN & BORUN LLP, 233 S. WACKER DRIVE, SUITE 6300, SEARS TOWER, CHICAGO, IL, 60606, US

(73) Assignee: IST INNOVATIVE SHOULDER TECHNOLOGY AG, Pfaffikon (CH)

(21) Appl. No.: 11/602,867

(22) Filed: Nov. 20, 2006

(30) Foreign Application Priority Data
Nov. 21, 2005 (EP) 05 025 324.4

(51) Int. Cl. A61B 17/58

(52) U.S. Cl. 606/73

(57) ABSTRACT

A bone anchor is provided having has a body with a proximal end and a distal end, a first section starting from the proximal end, with an outside thread and also a thread-free second section adjoining to it. The first section has a length that is adjusted to the thickness of the corticalis. The bone anchor has a device at the proximal end, the device extending from the proximal end into the body and serves to hold a holding thread.

### BACKGROUND OF THE INVENTION

The invention concerns a device for attaching soft tissues to a human or animal bone.

In various human and veterinary surgical processes, soft tissues, for example, tendons and ligaments, must be attached to a bone. The attachment can become necessary when, for example, due to an accident or a degenerative process, a tendon becomes separated from the bone and then the tendon will have to be attached to it again. In surgery various devices are used for this, which are frequently called bone anchors, as in the description below. The bone anchor is attached to an exposed bone in a preparatory step. With a suture material (for example, holding thread) attached to or guided into the bone anchor, for example, the tendon is attached to the bone anchor and thus to the bone.

A known bone anchor is the Statak® bone anchor made by the company Zimmer, Inc. This bone anchor has a cylindrical screw body with an outside thread, a distal wedge-shaped anchor tip and a proximal head part. The outside thread extends essentially between the anchor tip and the head part. The head part has a loop for holding the suture material perpendicular to the longitudinal axis of the screw body. Moreover, the head part has a multi-edged outer surface for placement of a tool. Therefore, the head part does not have any outside thread.

Another bone anchor is known from EP 829 233 A1. This bone anchor has a cylindrical screw body, which then goes to a distal part ending in a tip. An outside thread extends along the screw body between the distal part and the proximal end. Perpendicular to the longitudinal axis and within the part going to the tip, the bone anchor has a through bore to hold the suture material. In another example, instead of the through bore, the bone anchor has a notch starting from the distal part to hold the suture material. In both examples the screw body has two grooves opposite to one another, which extend parallel to the longitudinal axis and guide the suture material from the through bore or the notch to the proximal end. At the proximal end the screw body has an insertion slit for attaching a tool.

The above bone anchors are screw anchors, which are screwed into the bone. In addition to these screw anchors, striking anchors are used, which are driven into the bone with a special setting instrument. Wings or barbs extend from the cylindrical body of the bone anchor and these wedge themselves under the cortical bone and thus are supposed to keep the bone anchor safely in the bone. At the proximal end, the bone anchor, transversely to its longitudinal axis, has a lead-through to hold a holding thread. An example of such a bone anchor is the GII® anchor of DePuy Mitek, Inc.

Physicians, above all surgeons, expect that the bone anchors can be implanted reliably and be handled in a practical manner, for example, that the holding thread, if not already made available by the manufacturer, can be threaded easily, will not tear off accidentally, and will not become fouled. Moreover, the bone anchor must be held securely in the bone.

In the Statak® bone anchor, the head part has a double function, namely it serves both for holding the suture material and as a support for the tool. Due to the relatively small cross-section of the head part, there is a danger that the head part and thus the thread holder, will be screwed off during the setting of the bone anchor. Although in the bone anchor known from EP 829 233 A1 the insertion slit in the screw body is sunk so that the danger of it becoming screwed off no longer exists, the need to thread the thread into the groove from the tip to the proximal end seems not very practicable. Since the GII® anchor is not screwed in, in this anchor the problem of screwing the insertion slit off does not exist. However, in the case of this anchor the disadvantage is that the set anchor cannot be removed again or is difficult to remove because of the wedging of the barbs.

### SUMMARY OF THE INVENTION

Therefore, there is a need for a bone anchor of the type described at the outset with improved properties regarding reliability, ease of handling and secure anchoring in the bone.

Accordingly, a device for securing of soft tissues onto a bone that has a corticalis of a predetermined thickness, in a practical example, has a body with a proximal end and a distal end, which has a first section with an outside thread starting from the proximal end and then a second section following it which does not have any thread. The first section has a length that is adapted to the thickness of the corticalis of the bone. Moreover, this device has at the proximal end a device that extends from the proximal end into the body and serves to carry a holding thread.

As it will be evident from the description which follows and the drawings, the bone anchor described here can be conceived to be a striking anchor, which has an outside thread near the proximal end. As a result of this, the bone anchor can be set into the bone very rapidly. The outside thread fixes the bone anchor in the corticalis. In spite of that, if needed, it can be removed again relatively easily.

The device which is at the proximal end is designed so that it makes easier threading of the holding thread possible and that no edges are present at which the holding thread may tear. The device ends with the proximal end of the bone anchor, as a result of which the implanted bone anchor ends essentially at the outside surface of the bone. In this way, guiding of the holding thread over an edge of the bone and breaking of the thread there can be avoided.

### BRIEF DESCRIPTION OF THE FIGURES

Further embodiments, advantages, new properties and applications of the invention follow from the detailed description given below, with reference to the drawings. In the drawings, the same elements have the same reference numbers. The following are shown in the drawings:

FIG. 1 is a schematic side view of a practical example of a bone anchor;

FIG. 2 is a schematic cross-section through the bone anchor shown in FIG. 1 along a longitudinal axis;

FIG. 3 is a top view onto the bone anchor shown in FIG. 1; and

FIGS. 4 and 5 are schematic side views of other practical examples of a bone anchor.

### DETAILED DESCRIPTION

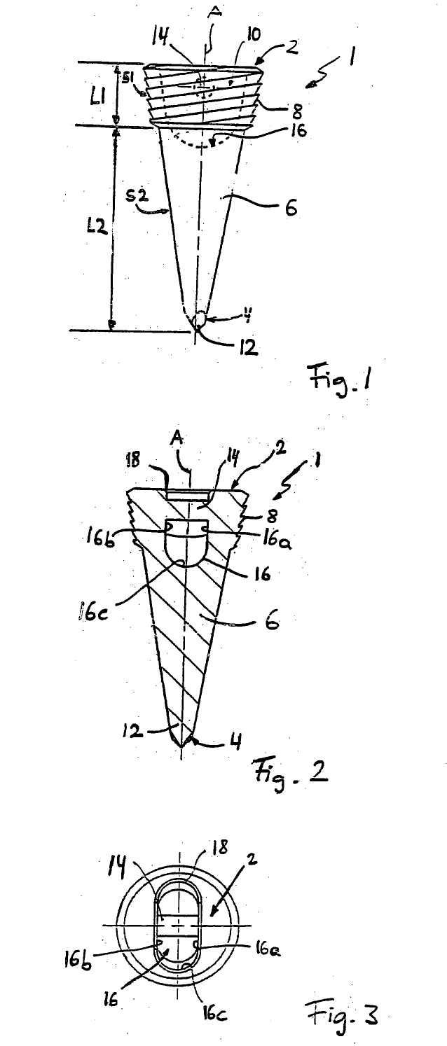

FIG. 1 shows a practical example of a bone anchor 1, which serves to secure soft tissues on a bone. The bone anchor 1 has a body 6 which extends symmetrically around a longitudinal axis A from a proximal end 2 to a distal end 4. Body 6 has a section S1 of a length L1, which, starting from proximal end 2 extends in the direction of the distal end 4. Section S1 has an outside thread 8. The body 6 also has a section S2 of a length L2, which is attached to section S1 and extends to the distal end 4. Section S2 does not have a thread.

As further explained below, the length L1 of section S1 is chosen so that the outside thread 8 screws into the corticalis of the bone. In a practical example, the length L1 is about 3 mm and the length L2 about 6 mm. The values for the length L1 may vary, but they correspond to the thicknesses that typically exist in humans in the corticalis. The total length of the bone anchor 1 can be about 5 to about 10 mm.

In the practical example shown in FIG. 1, the body 6 narrows from the proximal end 2 in the direction of the distal end 4. The body 6 can, for example, be cone-shaped or have the shape of a truncated cone. A narrowing or conical shape can be advantageous for impacting into the bone. However, it is understood that the bone anchor 1 in another practical example may have a different shape, for example, it may be cylindrical.

At distal end 4, the bone anchor 1 has a tip 12, which is joined to body 6. The tip 12 and body 6 in one practical example are made of one piece of material. The material is biocompatible and can be, for example, stainless steel, titanium, a titanium alloy or a resorbable material (for example polylactide or polyglyconate).

Moreover, the bone anchor 1 has a device 10, which extends from the proximal end 2 into body 6 and serves for holding the holding thread. In FIG. 1, for illustration, the device 10 is indicated by dashed lines, since it is not visible when viewing the bone anchor 1 from the side. In the case of the bone anchor 1 shown, in the area of the proximal end 2, the device 10 forms a cavity, which is delineated by a wall surface 16 and is open at the proximal end 2. The wall surface 16 has a concave shape in FIG. 1. In addition, the device 10 has a bridge 14 with a circular cross-sectional surface that extends into the cavity.

FIG. 2 shows a schematic cross-section through the bone anchor 1 shown in FIG. 1 along longitudinal axis A. In the practical example shown in FIG. 2 the wall surface 16 has curved surface section 16c and two side surfaces 16a, 16b, which extend parallel to the longitudinal axis A and go into the flat section 16c. The wall surface 16 forms the hollow cavity in the practical example shown so that a disk-shaped hollow cavity is produced. It is understood that in other practical examples the cavity may have another shape, for example, the shape of a half-oval or a half-sphere.

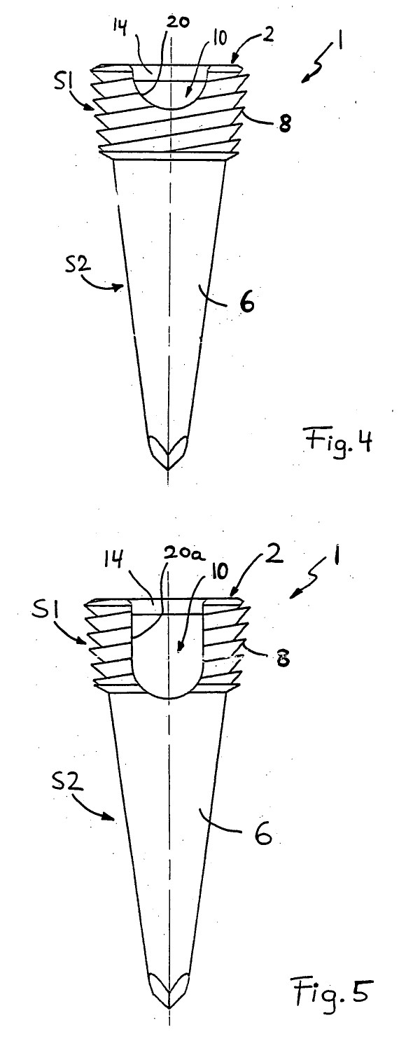

The bridge 14 extends essentially transversely to the longitudinal axis A between side parts 16a, 16b. Hereby in a practical example, bridge 14 is located on the level of the plane of the proximal end 2 as shown, for example, in FIGS. 4 and 5. In the representation shown in FIG. 2, the bridge 14 is set back from the plane of the proximal end 2 in the direction of the cavity. In a practical example, the bridge 14 is set back less than about 1 mm from the proximal end 2. The transition from the plane of the proximal end 2 into the cavity is rounded or inclined in a region 18 in order to avoid edges at which the holding thread could be separated.

FIG. 3 shows a top view onto the bone anchor 1 shown in FIG. 1. One can see the proximal end 2, the cavity formed by wall surface 16 and the bridge 14 extending into the cavity. In the top view, the cavity has an elongated oval opening, which is formed by side surfaces 16a, 16b and flat section 16c. The bridge 14 divides the oval opening into two approximately equally-sized partial openings. For example, a surgeon can guide an attaching thread through one partial opening into the cavity, guide it around the bridge 4 and then remove it through the other partial opening.

At the proximal end 2 the surgeon can also set a tool in order to screw the bone anchor 1 into the bone. In a practical example, the tool is designed in such a way that it extends into the cavity. The tool can have a recess corresponding to the diameter of bridge 14 in order to guide the tool through bridge 14 into the cavity, so that the torque can grip in the inside of body 6. The danger of damaging the bone anchor 1 is thereby significantly reduced.

FIGS. 4 and 5 show schematic side views of other practical examples of a bone anchor 1. In these practical examples, in one or several areas of section S1 there is no anchor material and thus no outside thread 8 present. For example, from the bone anchor 1 shown in FIGS. 1-3, the anchor material can be removed over a length of several thread turns (can be ground off). As a result of this, near the proximal end 2 less anchor material is present that would displace bone material. As a result of this, the stress can be removed from the bone material in this area.

In the practical example of bone anchor 1 shown in FIG. 4, in an area 20, the anchor material has been removed starting from the proximal end 2 over approximately two thread turns, so that a side opening is produced through which the bridge 14 and a part of the cavity formed by side surface 16 are visible. On an opposite side of the opening, the anchor material can also be removed in the same way, as a result of which another side opening is produced.

In the practical example of bone anchor 1 shown in FIG. 5, on the other hand, in an area 20a the anchor material was removed over the entire length of section S1. The bridge 14 and a part of the cavity formed by wall area 16 are also thereby visible through a side opening formed. In this practical example too, the anchor material can be removed in the same way on the opposite side of the opening, as a result of which another side opening is formed.

The bone anchor 1 that is described above with the aid of practical examples, makes handling possible in a practical and reliable way, since the device which holds and guides the holding thread at the proximal end 2 is easily accessible to the surgeon and in the implanted state it essentially ends at the bone surface. In this practical example the threaded holding thread is not guided over a bone edge where it could tear. The concave wall area 16 supports the surgeon during threading of the holding thread. The side openings shown in FIGS. 4 and 5 additionally facilitate the threading of the holding thread. The device 10 and especially bridge 14 are designed in such a way that they do not have any sharp edges on which the holding thread could tear.

In addition, bone anchor 1 ensures secure holding in the bone. This is achieved above all by the fact that the outside thread 8 extends in the region of the proximal end 2 along section S1. Thus the outside thread 8 finds good holding in the corticalis of the bone and not in the region of the bone marrow, where the section S2, that has no threads, sits. In the case of patients in whom little structure is present in the region of the bone marrow, for example, in elderly patients, as a result of this, secure holding in the bone can be achieved.

The bone anchor 1 described above with the aid of practical examples can be struck directly into the bone. Only then is the bone anchor 1 pushed further into the bone by screwing it in, whereby the section S1 comes to lie essentially in the corticalis.

Depending on the practical example, the outside thread 8 does not have to extend to the outermost edge of the proximal end 2, although this is preferred; however, it extends so close to the proximal end 2 that the function of the anchoring can be fulfilled by the outside thread 8 in the corticalis.

### REFERENCE LIST

A Axis
S1 Section (length L1)
S2 Section (length L2)
1 Bone anchor
2 Proximal end
4 Distal end
6 Body
8 Outside thread
10 Device
12 Tip
14 Bridge
16 Wall surface
16a Side surface
16b Side surface
16c Surface section
18 Region
20 Region
20a Region

1. A device (1) for securing soft tissues on a bone, having a thickness predetermined by the corticalis,
which has a body with a proximal end and a distal end and which has a first section with an outside thread starting from proximal end and a section following this which does not have a thread, whereby the first section has a length that is adapted to the thickness of the corticalis; and
which has a device 4 at the proximal end which extends from the proximal end into body and serves to hold a holding thread.

2. The device according to claim 1, in which there is a tip at the distal end of the body.

3. The device according to claim 1X, in which the device has a bridge within the body, the bridge separating the device into a first and second part, whereby the holding thread can be threaded from the first part to the second part.

4. The device according to claim 3, in which the bridge has a circular cross-section.

5. The device according to claim 3, in which the bridge is arranged near the proximal end.

6. The device according to claim 1, in which the device has a concave wall surface in the body.

7. The device according to claim 1, in which a transition of a plane of the proximal end in the direction of the device is rounded or inclined within a region.

8. The device according to claim 1, in which the body narrows from the proximal end in the direction of the distal end.

9. The device according to claim 1, in which anchor material is removed from the first section in at least one region so that a side opening is produced.
